SEQUENCE LISTING (13 sequences)

SEQ ID NO: 1 - DNA, 34 bp, Unknown Organism
  Description of Unknown Organism Nonhuman mammal nucleotide sequence
ataacttcgtatagtatacattatacgaagttat

SEQ ID NO: 2 - DNA, 34 bp, Unknown Organism
  Description of Unknown Organism Nonhuman mammal nucleotide sequence
acaacttcgtataatgtatgctatacgaagttat

SEQ ID NO: 3 - DNA, 34 bp, Unknown Organism
  Description of Unknown Organism Nonhuman mammal nucleotide sequence
gaagttcctattctctagaaagtataggaacttc

SEQ ID NO: 4 - DNA, 34 bp, Unknown Organism
  Description of Unknown Organism Nonhuman mammal nucleotide sequence
ataacttcgtatagcatacattatacgaagttat

SEQ ID NO: 5 - DNA, 44 bp, Unknown Organism
  Description of Unknown Organism Nonhuman mammal nucleotide sequence
gaacctcactttcagaagattcaagagatcttctgaaagtgagg

SEQ ID NO: 6 - DNA, 23 bp, Unknown Organism
  Description of Unknown Organism Nonhuman mammal nucleotide sequence
ctccctatcagttgatagagaaa

SEQ ID NO: 7 - DNA, 22 bp, Unknown Organism
  Description of Unknown Organism Nonhuman mammal nucleotide sequence
ttatccagccctcactccttct

SEQ ID NO: 8 - DNA, 22 bp, Unknown Organism
  Description of Unknown Organism Nonhuman mammal nucleotide sequence
cattaagggatcagttatctag

SEQ ID NO: 9 - DNA, 20 bp, Unknown Organism
  Description of Unknown Organism Nonhuman mammal nucleotide sequence
ccttgggttcaggggattat

SEQ ID NO: 10 - DNA, 20 bp, Unknown Organism
  Description of Unknown Organism Nonhuman mammal nucleotide sequence
ggcttcaggatgtccatgtt

SEQ ID NO: 11 - DNA, 22 bp, Unknown Organism
  Description of Unknown Organism Nonhuman mammal nucleotide sequence
atggcacccaagaagaagagga

SEQ ID NO: 12 - DNA, 22 bp, Unknown Organism
  Description of Unknown Organism Nonhuman mammal nucleotide sequence
ctaatcgccatcttgcagcagg

SEQ ID NO: 13 - DNA, 33 bp, Unknown Organism
  Description of Unknown Organism Nonhuman mammal nucleotide sequence
acacctggagagaagctttcaacgagactaacc

(19) United States
(12) Patent Application Publication
(10) Pub. No.: US 2005/0235370 A1
(43) Pub. Date: Oct. 20, 2005

(54) Spatiotemporally controlled adult somatic mutagenesis system

(75) Inventors: Winston C. Y. Yu, Jhunan Township (TW); Hung-Shu Chang, Jhunan Township (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: National Health Research Institutes

(21) Appl. No.: 11/010,124

(22) Filed: Dec. 9, 2004

Related U.S. Application Data
(60) Provisional application No. 60/529,409, filed on Dec. 12, 2003.

(51) Int. Cl. A01K 67/027; C12Q 1/68; C12N 15/09

(52) U.S. Cl. 800/14; 435/320.1; 435/325

(57) ABSTRACT

The present invention relates to an inducible and reversible gene knockdown system. The target gene is under the control of a suppressor which is regulated by a repressible promoter with the repressible promoter being under the control of a transcription-activating gene to activate gene expression and a transcriptional repressor to repress gene expression. Furthermore, the repressible promoter is spatial specific and the transcriptional repressor can be temporally controlled, which lead to a spatiotemporal gene knockdown system.

This application claims the benefit of U.S. Provisional Application No. 60/529,409, filed Dec. 12, 2003, which is incorporated herein by reference.

### BACKGROUND OF THE INVENTION

1. Field of Invention

This invention relates generally to transgenic animals with temporally controlled spatial specific gene knockdown system and more particularly to a gene knockdown system with a target gene suppressor regulated by both a transcription-activating gene and a transcriptional repressor.

2. Description of Related Art

One of the ultimate goals of the human genome project is to identify the biological functions of genes and their relationships with genetic diseases. Gene functions are recognized either directly by appropriate assays or inferred by studying homologous genes in other species using transgenic or knockdown techniques (Hadjantonakis A. K. et al., (2003) Nat. Rev. Genet. 4: 613-625).

Transgenic animal models using specific promoters have provided a wealth of information about the function of specific genes (Udvadia A. J. & Linney E. (2003) Dev. Biol. 256: 1-17; Stricklett P. K. (1999) Am. J. Physiol. 276: F651-657). However, the limitations of promoter-controlled transgene expression have become clear.

The constitutive system of the promoter-controlled gene expression precludes control over the timing of the expression, which depends entirely on the properties of the promoters used. The promoters in this setting are active constitutively, many starting early in embryonic stage. If the transgene product happens to be toxic to the organism, it can be highly detrimental to the developing embryo. Also, if the defect of target gene is important to embryonic development, it may have a lethal impact during embryogenesis. Therefore, a constitutive over-expression transgenic system is not suitable for answering the questions concerning specific genetic defects and their relationship to particular specific proteins.

To address the limitations of the constitutive over-expression system, many laboratories have invested tremendous amounts of time and effort in establishing conditional or inducible transgenic modeling systems. Several different inducible systems are currently in use, including Mfp, tetracycline and Cre-LoxP systems (Stricklett P. K. (1999) Am. J. Physiol. 276: F651-657; Acres B. et al. (2000) Cancer Immunol., Immunother. 48: 588-94; Zuo J. (2002) J. Neurobiol. 53: 286-305; Ryding A. D. et al. (2001) J. Endocrinology. 171: 1-14). However, there are problems with these systems. For example, not only is the knockout system of Cre-LoxP difficult to construct and expensive to maintain in stem cells, but it also has a high frequency of embryonic fatality and a low germline transmission efficiency.

In the development of specific gene knockout techniques, a significant finding in recent years has been the discovery of RNA interference (RNAi). RNAi involves double-stranded RNA of 21-23 nucleotides that specifically and post-transcriptionally silence a gene. It has been adopted as a tool and used in a variety of functional genomic projects in a wide range of species. It has also been adapted for high-throughput use in the transient knockdown of gene expression in cell lines and animals (Hannon G J., (2002) Nature 418: 244-251. Until recently, three major methods have been used to synthesis the RNA duplex of 21-23 nucleotides for RNAi: chemical synthesis; in vitro double stranded (ds)/hairpin-structure RNAs transcription; and in vivo ds/hairpin-structure transcription from a plasmid or vector. However, the polymerase III promoters, U6 or H3, of RNAi are active in all tissues and cannot be used to generate tissue-specific knockout mice. In addition, all the RNA duplexes synthesized have the limitations of being transient and non-heritable (Caplen N.J., (2003) Expert. Opin. Biol. Ther. 3: 575-586).

Therefore, there are many limitations in current approaches in creating gene knockdown animal models, for example the use of recombinase or RNAi.

A more precisely controlled, and even reversible, system is desired to overcome many of these limitations.

### SUMMARY OF THE INVENTION

The present invention addresses these limitations by providing a method for producing a reversible-gene knockdown system comprising the introduction into a nonhuman mammal a nucleic acid comprising the coding sequence for at least one target gene suppressor. In one embodiment, the target gene suppressor is siRNA. The expression of the target gene suppressor may be regulated by a first repressible promoter regulated by at least one transcription-activating gene, such as a transposon or site-specific DNA recombinase, and the expression of the transcription-activating gene is itself regulated by, at least, a second repressible promoter under the control of a transcriptional repressor. Conversely, the expression of the target gene suppressor is regulated by the first repressible promoter under the control of the same transcriptional repressor. The repressible promoter may be spatial-specific and/or tissue-specific. In addition, the transcriptional repressors may be under temporal control.

The present invention also provides a method for producing an inducible-gene knockdown system. This system comprises the introduction into a nonhuman mammal a nucleic acid comprising the coding sequence for at least one target gene suppressor, which may be siRNA, as above. The expression of the target gene suppressor is regulated by a repressible promoter regulated by at least one transcription-activating gene, which may be a transposon or a site-specific DNA recombinase, as above. The expression of the transcription-activating gene is regulated by a repressible promoter under the control of at least one transcriptional repressor. As above, the repressible promoter may be spatial-specific or tissue specific. Also as above, the transcriptional repressor may be under temporal control.

In addition, the invention is directed to a method for producing a reversible gene knockdown system by introducing into a nonhuman mammal a nucleic acid comprising a coding sequence for at least one target gene suppressor regulated by a repressible promoter which is under the control of at least one transcription-activating gene and at least one transcriptional repressor. The transcription-activating gene is regulated by an inducible promoter. The inducible promoter may be one of the many types of stress promoter, such as a heat promoter.

Furthermore, the invention also contemplates a method for producing an inducible gene knockdown system comprising introducing into a nonhuman mammal a nucleic acid comprising a coding sequence for at least one target gene suppressor wherein expression of the at least one target gene suppressor is regulated by a promoter controlled by at least one transcription-activating gene, and the transcription-activating gene is regulated by an inducible promoter, which can be a stress promoter. The stress promoter can be a heat shock promoter.

The present invention also provides the non-human mammals crated by the above-described methods as well as the associated recombinant DNA constructs and transfected cells.

### BRIEF DESCRIPTION OF THE DRAWINGS

The patent or application file contains at least one drawings executed in color. Copies of this patent or patent application publication with color drawing(s) will be provided by the Office upon request and payment of the necessary fee.

FIG. 1 shows schematic diagrams of SIRIUS-ABCA1, AlbTet-Cre and the resulting reSIRIUS-ABCA1 construction.

FIG. 2 shows (a) result of PCR analysis of recombined reSIRIUS-ABCA1 after treatment with Cre recombinase, (b) fluorescence pictures of transfected cells with different vectors and results of Western blot analysis of Cre recombinase and PCR analysis of reSIRIUS-ABCA1, and (c) result of Western blot analysis in the detection of ABCA1 protein.

FIG. 3 shows (a) separate lines of transgenic animals carrying AlbTet-Cre and SIRIUS-ABCA1 plasmid respectively and their offsprings, (b) result of PCR analysis of reSIRIUS-ABCA1 and Northern blot analysis of ABCA1 expression, (c) result of Oil Red O staining of lipid in the livers derived from non-induced and induced mice.

FIG. 4 shows schematic diagrams of SIRIUS-Tet and AlbTet-Cre constructions.

### DETAILED DESCRIPTION OF THE INVENTION

The present invention provides a method for producing a reversible gene knockdown transgenic animal. Conversely, the invention is also directed to a method for producing an inducible gene knockdown transgenic animal. The invention further includes the genetic material employed to create such gene knockdown systems, and the transgenic animals created thereby. More importantly, the gene knockdown system is under both spatial and temporal control.

Definitions

As used here, the term “knockout” or “knockdown”, used interchangeably, refers to an organism that has a normal functional gene replaced by a non-functional form of the gene, with the function of that particular gene eliminated. Gene “knockout” produces model systems for studying inherited human diseases, investigating the nature of genetic diseases and the efficacy of different types of treatment, and for developing effective gene therapies to cure these diseases. For example, a “knockout” line of mutant mice homozygous for a null allele of the cystic fibrosis transmembrane regulator gene demonstrates symptoms similar to those of humans with cystic fibrosis. These mice provide a model system for studying this genetic disease and developing effective therapies.

As used herein, the term “reversible gene knockdown system” means a model where the initiation or level of expression of a gene is regulatable to be activated or repressed.

As used herein, the term “inducible gene knockdown system” refers to a model where the initiation or level of expression of a gene is regulated and induced by a molecule.

As used herein, the term “nonhuman mammal” includes rodents, rats, mice, non-human primates, sheep, dogs, cows, chickens, etc.

As used herein, the term “target gene suppressor” is a nucleic acid the expression of which directly or indirectly suppresses the expression of another gene of interest, including but not limited to interfering RNAs, siRNAs, tumor suppressor gene, eukaryotic trans-dominant suppressor gene, and any of a number of suppressor gene which are known in the art. The term “eukaryotic trans-dominant suppressor gene” refers to a gene encoding a polypeptide translation product capable of suppressing the activity of a eukaryotic protein that requires an oligomeric state for function, by forming an inactive oligomer with a wildtype subunit of the protein and thereby preventing that wildtype subunit from forming an active dimer with a second wildtype subunit. The methods are particularly useful for producing a trans-dominant suppressor gene that encodes an inactive subunit of a eukaryotic growth factor and thus suppresses the activity of that growth factor.

As used herein, the term “siRNA” refers to double or single stranded small interfering RNA molecules which mediates the RNA interference (RNAi) cellular mechanism that regulates the expression of genes. RNA interference is used to block the function of the endogenous gene/protein and thus mimic the effect of a loss of function mutation. Methods for using RNAi, either exogenous addition or transcription in vivo, are known in the art (see Schubiger and Edgar, Methods in Cell Biology (1994) 44:697-713, and PCT application WO 99/32619, respectively.

As used herein, the term “ABCA1 siRNA” refers to the siRNA with sequences designed to suppress the expression of the ABCA1 gene (ATP-binding cassette subfamily A member), the gene found to relate to Tangier disease, a congenital absence of serum α-lipoprotein resulting in the accumulation of cholesteryl esters in tissues.

As used herein, the term “promoter” is used in its conventional sense to refer to a nucleotide sequence at which the initiation and rate of transcription of a coding sequence is controlled. The promoter contains the site at which RNA polymerase binds and also contains sites for the binding of regulatory factors (such as repressors or transcription factors). Promoters can be naturally occurring or synthetic. The promoters can be endogenous to the virus or the host or derived from other sources. The promoter can be constitutively active, or temporally controlled (temporal promoters), activated in response to external stimuli (inducible), active in particular cell type or cell state (selective). The term also covers so-called “leaky” promoters, which regulate expression of a selected DNA primarily in one tissue, but cause expression in other tissues as well.

As used herein, the term “repressible promoter” means a promoter that contains site(s) for the binding of a regulatory factor, a repressor, that represses the controlling function of the promoter on the initiation and rate of transcription of a coding sequence.

As used herein, the term “transcription-activating gene” means a nucleotide sequence encoding a factor that initiates transcription wherein the binding of the factor to specific DNA sites activates transcription via direct or indirect physical association or by causing an effect or a cascade of effects leading to transcription, including but not limited to a transcriptional activator, site specific DNA recombinase or transposon.

As used herein, the term “site specific DNA recombinase” refers to an enzyme which catalyzes the exchange of DNA segments at specific recombination sites, and when two specific recombination sites are placed on a DNA fragment flanking a particular nucleotide sequence of interest, the enzyme catalyzed exchange of DNA fragments can lead to an excision of the nucleotide sequence of interest. An example of site-specific recombinase include, but are not limited to, bacteriophage P1 Cre recombinase, yeast FLP recombinase, and any of a number of recombinases which are known in the art to have similar functions.

As used herein, the term “transposon” encompasses a segment flanked by particular cis-acting sites that are required for mobilization to occur, together with the genes that specify the proteins that act on those cis-acting sites to mobilize the segment defined by them, whether or not the protein-encoding genes lie between the sites mentioned. This transposon thus provides the protein products required for mobilization. However, an entire transposon is not necessary to practice the invention. Thus, the term “transposon” as used herein can also refer to DNA minimally comprising the cis-acting sites at which the trans-acting proteins act to mobilize the segment defined by the sites. It is also understood that the sites may contain intervening DNA. Although transposons can be divided into subgroups based on their transposition mechanism, they all have similar DNA element structures (Orle, K. and Craig, N., Gene 1991, 104, 125-131). Transposons in their simplest form carry at least two genes. Typically, one gene codes for an antibiotic resistance factor and the second gene encodes one or more transposases. The transposase is an enzyme responsible for the recognition of the transposon DNA element, the insertion site on the target DNA, and for catalyzing the transposition event. Transposons are responsible for various types of genetic rearrangements, including chromosome breakage, deletions, duplications, inversions and translocations are derived from natural gene transfer vectors in bacteria, yeast, Drosophila melanogaster and other organisms Transposons are segments of double-stranded DNA, made up of some thousands of nucleotides. Insertion of a transposon into a gene which specifies the amino acid sequence of an enzymically active protein causes complete loss of ability to synthesize that protein in active form. However when the whole transposon is deleted or excised from the gene into which it was inserted, this gene in consequence is being restored to its original state, so that it again specifies an enzymically active protein.

As used herein, the term “at least one transcriptional repressor” refers to proteins or coeffector domains of proteins that binds to a repressible promoter to inhibit transcription of a gene, and where there are more than one transcriptional repressor, the transcriptional repressors may be the same or different. There can be a first transcriptional repressor and a second transcriptional repressor. The “first transcriptional repressor” refers to at least one transcriptional repressor for acting on at least one repressible promoter, and if there are more than one transcriptional repressors, they can be the same or different transcriptional repressors, and the “second transcriptional repressor” refers to at least one other transcriptional repressor acting on at least one other repressible promoter, and if there are more than one transcriptional repressors, they can be the same or different transcriptional repressors.

As used herein, the term “induction agent” refers to a molecule that initiates or increases gene transcription.

As used herein, the term “induction agent regulated repressor” refers to a repressor inducible by exposure to an environmental inducing agent. Appropriate environmental inducing agents include exposure to heat, various steroidal compounds, divalent cations (including Cu.sup.+2 and Zn.sup.+2), galactose, tetracycline, IPTG (isopropyl .beta.-D thiogalactoside), as well as other naturally occurring and synthetic inducing agents and gratuitous inducers. It is important to note that, in certain modes of the invention, the environmental inducing signal can correspond to the removal of any of the above listed agents which are otherwise continuously supplied in the uninduced state, such as the rTA based system.

As used herein, the term “control”, “controlled” or “under the control” and “regulate” or “regulated” means being under the command via direct or indirect physical association or via causing an effect or a cascade of effects thereon.

As used herein, the term “spatial-specific” refers to gene expression in a spatial pattern, which may be obtained by in-situ hybridization of immuno-histochemical markers and are characterized by profiles of locations of gene activation (Costa, L. F. (2003) Business Briefing: Pharmatech 81).

As used herein, the term “tissue-specific” means gene expression found only in certain tissue.

As used herein, the term “liver-specific” means gene expression found in liver cells.

As used herein, the term “under temporal control” means control exerted in a time dependent manner according to a predetermined time.

As used herein, the term “delivery or removal” means to put forth, to come in contact with, or to take away.

As used herein, the term “an exogenous molecule” means one or more molecule, i.e. proteins, nucleic acids, and chemical compounds, which is not normally produced by the animal. Alternatively, the molecule may be produced by the animal but is not normally under the control of a regulatory sequence of an endogenous gene.

As used herein, the term “Cre” means Cre recombinase.

As used herein, the term “Cre recombinase” refers to the site-specific DNA recombinase derived from the P1 bacteriophage. Cre recombinase is a 38-kDa product of the cre (cyclization recombination) gene of bacteriophage P1 and is a site-specific DNA recombinase of the nt family (Sternberg, N. et al. (1986) J. Mol. Biol. 187:197-212). Cre recombinase recognizes a 34-bp site on the P1 genome called loxP (locus of X-over P1, loxP site) and efficiently catalyzes reciprocal conservative DNA recombination between pairs of loxP sites. The loxP site comprises two 13-bp inverted repeats flanking an 8-bp nonpalindromic core region. Cre recombinase-mediated recombination between two directly repeated loxP sites results in excision of DNA between. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; and Shaikh et al. (1977) J. Biol. Chem. 272:5695-5702. The Cre recombinase will recognize a number of variant or mutant lox sites relative to the loxP sequence. Conversely, the loxP sequence can be recognized by mutants and variants of Cre recombinase. Examples of these variant or mutant Cre recombination sites include, but are not limited to, the loxB, loxL and loxR sites which are found in the E. coli chromosome (Hoess et al. (1982) Proc. Natl. Acad. Sci. USA 79:3398). Other variant lox sites include: loxP511 site (5′-ATAACTTCGTATAGTATACATTATACGAAGTTAT-3′ (Hoess et al. (1986) Nucleic Acid Res. 14:2287-2300)) and loxC2 site (5′-ACAACTTCGTATAATGTATGCTATACGAAGTTAT-3′ (U.S. Pat. No. 4,959,317)). Therefore, the term “Cre recombinase” includes Cre recombinase and all mutants and variants thereof with the recombination catalyzing function described above, and the term “loxP site” includes the 34 bp site described above and any mutants and variants recognizable by the Cre recombinase, including its variants.

As used herein, the term “Flp” means FLP recombinase.

As used herein, the term “FLP recombinase” refers to a site-specific DNA recombinase derived from yeast, the 2pi plasmid of Saccharomyces cerevisiae, that recognizes a 34 base pair DNA sequence, termed the “FRT site” (FLP recombinase target). “FLP” is a 423 amino acid protein capable of binding to FRT recombination target sites and mediating conservative site-specific recombination between FRT sites. The basic configuration of the FRT site comprises a 48 nucleotide DNA sequence consisting of an 8-base-pair core and three 13-base-pair symmetry elements where two symmetry elements occur in direct orientation on the 5′ end of the core sequence and the third element occurs in inverted orientation on the 3′ end of the core sequence. The FRT site has also been identified as minimally comprising two 13 base-pair repeats, separated by an 8 base-pair spacer, as follows: 5′-GAAGTTCCTATTC[TCTAGAAA]GTATAGGAACTTC-3′XbaI site. The nucleotides in the above “spacer” region can be replaced with any other combination of nucleotides, so long as the two 13 base-pair repeats are separated by 8 nucleotides. The actual nucleotide sequence of the spacer is not critical, although those of skill in the art recognize that, for some applications, it is desirable for the spacer to be asymmetric, while for other applications, a symmetrical spacer can be employed. It is also recognized by one skilled in the art that modified forms or mutants of FLP recombinase can recognize the FRT site and its variants. Therefore, the term “FLP recombinase” and “FRT site” includes all variants and mutants carrying out their functions as described above.

As used herein, the term “tetracycline analog” refers to molecules that mimic the effects of tetracycline. Examples of tetracycline analogs include but not limited to anhydrotetracycline, doxycycline, chlorotetracycline, epioxytetracycline and the like.

As used herein, the term “tetracycline regulated repressors” means transcriptional repressors effected by the presence or absence of tetracycline.

As used herein, the term “tetracycline analog regulated repressors” means transcriptional repressors effected by the presence or absence of tetracycline analog.

As used herein, the term “cell” means primary cells and cultured cells.

As used herein, the term “transfected” means the acquisition of new genetic markers by incorporation of added DNA.

As used herein, the term “transgene” means a nucleic acid sequence (encoding, e.g., one of the polypeptides, or an antisense transcript thereto) which has been introduced into a cell. A transgene could be partly or entirely heterologous, i.e., foreign, to the transgenic animal or cell into which it is introduced, or, homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

As used herein, the term “recombinant DNA”, refers to the various component domains or sequences that are mutually heterologous in the sense that they do not occur together in the same arrangement in nature. More specifically, the component portions are not found in the same continuous nucleotide sequence in nature, at least not in the same order or orientation or with the same spacing present in the recombinant DNA molecule of this invention. Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989. “recombinant DNA construct” is a construct of recombinant DNA, including but not limited to vectors.

As used herein, the term “U6 promoter” refers to a promoter which is able to initiate transcription by RNA polymerase III from a position upstream of the transcribed DNA. U6 promoters have been referred to in the literature, in at least one instance, as RNA polymerase III, type III promoters (Willis, I. (1993) FEBS 212: 1-11). The U6 promoter contains regulatory elements which are necessary and sufficient to facilitate transcription by RNA polymerase III, but these regulatory elements are not themselves transcribed. Thus, U6 RNA polymerase III promoters include the following promoters: naturally-occurring U6 from higher order eukaryotes (Das et al. (1988) EMBO J. 7 (2): 503-512), 7SK (Murphy et al. Cell 51:81-87), H1 RNA gene (Hannon, G et al. (1991) J. Biol. Chem. 266 (34): 22796-22799), U3 snRNA genes in plants (Marshallsay C. et al. (1992) Plant Molecular Biology 19 (6): 973-983), and MRP gene (Yuan, Y. and Reddy, R. Biochem. et Biophys. Acta 1089 (1): 33-39), as well as any recombinant promoter sequence which is able to initiate transcription by RNA polymerase III without itself being transcribed.

As used herein, the term “GFP” means Green Fluorescent Protein. GFPs are involved in bioluminescence in a variety of marine invertebrates, including jellyfish such as Aequorea spp. (Morise, H., et al., Biochemistry 13:2656-2662 (1974); Prendergast, F. G., and Mann, K. G, Biochemistry 17:3448-3453 (1978); Ward, W. W., Photochem. Photobiol. Rev. 4:1-57 (1979) and the sea pansy Renilla reniformis (Ward, W. W., and Cormier, M. J., Photochem. Photobiol. 27:389-396 (1978); Ward, W. W., et al., Photochem. Photobiol. 31:611-615 (1980)). The GFP isolated from Aequorea victoria has been cloned and the primary amino acid structure has been deduced (Prasher, D. C., et al., Gene 111:229-233 (1992). The chromophore of A. victoria GFP is a hexapeptide composed of amino acid residues 64-69 in which the amino acids at positions 64-67 (serine, tyrosine and glycine) form a heterocyclic ring (Prasher, D. C., et al., Gene 111:229-233 (1992); Cody, C. W., et al., Biochemistry 32:1212-1218 (1993)). Resolution of the crystal structure of GFP has shown that the chromophore is contained in a central .alpha.-helical region surrounded by an 11-stranded beta.-barrel (Ormo, M., et al., Science 273:1392-1395 (1996); Yang, F., et al., Nature Biotech. 14:1246-1251 (1996)). Upon purification, native GFP demonstrates an absorption maximum at 395 nanometers (nm) and an emission maximum at 509 nm (Morise, H., et al., Biochemistry 13:2656-2662 (1974); Ward, W. W., et al., Photochem. Photobiol. 31:611-615 (1980)) with exceptionally stable and virtually non-photobleaching fluorescence (Chalfie, M., et al., Science 263:802-805 (1994)). While GFP has been used as a fluorescent label in protein localization and conformation studies (Heim, R., et al., Proc. Natl. Acad. Sci. USA 91:1250-1254 (1994); Yokoe, H., and Meyer, T., Nature Biotech. 14:1252-1256 (1996)), it has gained increased attention in the field of molecular genetics since the demonstration of its utility as a reporter gene in transfected prokaryotic and eukaryotic cells (Chalfie, M., et al., Science 263:802-805 (1994); Heim, R., et al., Proc. Natl. Acad. Sci. USA 91:1250-1254 (1994); Wang, S., and Hazelrigg, T., Nature 369:400-403 (1994)).

As used herein, the term “EGFP” means Enhanced Green Fluorescent Protein which is a new version of GFP developed via mutation. It is a “humanized” GFP DNA, the protein product of which enjoys increased synthesis and improved folding in mammalian cells (see Cormack, B. P., Valdivia, R. H., and Falkow, S. (1996) Gene 173, 33-38; Haas, J., Park., E. C., and Seed, B. (1996) Current Biology 6, 315-324; and Yang, T. T., Cheng, L., Kain, S. R. (1996) Nucleic Acids Research 24, 4592-4593).

As used herein, the term “operably linked” means two polynucleotides arranged within the nucleic acid moiety in such a manner that at least one of the two polynucleotides is able to exert a physiological effect by which it is characterized upon the other. For example, a promoter operably linked to the coding region of a gene is able to promote transcription of the coding region.

As used herein, the term “inducible promoter” means a promoter that can be induced by the binding of a regulatory factor or any form of external stimuli leading to the transcription of the gene operably linked thereto.

As used herein, the term “stress promoter” refers to an inducible promoter induced by external environmental stress, including but not limited to arsenite treatment, anaerobic condition, heat-shock, cold-shock.

As used herein, the term “heat-shock promoter” means an inducible promoter induced by sudden elevation of temperature leading to the initiation or increased expression of a protein.

Methods for Producing Transgenic Gene Knockdown Animal

The present invention provides in a first aspect a method for producing a reversible gene knockdown system by introducing into a nonhuman mammal a nucleic acid molecule that comprises a coding sequence for a target gene suppressor, and a first repressible promoter for regulating the expression of the target gene suppressor. Regulation of the target gene suppressor by the first repressible promoter is controlled by a transcription-activating gene. The first repressible promoter is also under the control of a transcriptional repressor. The transcription-activating gene is regulated by a second repressible promoter under the control of the same transcriptional repressor. There can always be one or more target gene suppressor with one or more corresponding repressible promoter, transcription-activating gene, and transcriptional repressor. Alternatively, there can be one or more regulating sequences, i.e. transcription-activating gene and transcriptional repressor etc., for the same target gene suppressor.

The instant gene knockdown model employs a gene suppressor to suppress the expression of a specific gene of interest, i.e. a target gene suppressor. This target gene suppressor can act at the transcriptional or translational level, and its product can act directly or indirectly on the target gene. One example of a gene suppressor is the post-transcriptional gene silencing mechanism mediated by interference RNA. Any gene encoding an interference RNA or small interference RNA (siRNA) targeting a specific gene can lead to the degradation of the mRNA of the targeted gene and thus suppress gene expression. Also, tumor suppressor gene and eukaryotic trans-dominant suppressor gene encodes polypeptides that act on the target gene or its product to prevent expression. Fundamentally, the target gene suppressor can be any gene that functions to suppress gene expression of another gene or any of a number of suppressor genes that are known in the art.

The promoter for the target gene suppressor contains an element for binding by a transcriptional repressor inducible by an induction agent. Binding by the transcriptional repressor on the promoter sequence of the target gene suppressor leads to inhibition of transcription. Therefore, it is a “repressible promoter”. The promoter for the transcription-activating gene is also referred to as a “repressible promoter” for the same reason that it can be bound by a transcriptional repressor to repress transcription of the transcription-activating gene.

There can be just a single locus containing the coding sequence for the transcriptional repressor that will bind to promoters of both the transcription-activating gene and the target gene suppressor. Alternatively, there can be different loci containing coding sequences for transcriptional repressors that bind to the promoters for the transcription-activating gene and the target gene suppressor. In particular, there can be one or more transcriptional repressor coding sequences for the transcription-activating gene and one or more transcriptional repressor coding sequences for the target gene suppressor. The transcriptional repressor coding sequences for the transcription-activating gene and the target gene suppressor may be the same or different, i.e. inducible by the same or different induction agent.

The transcription-activating gene can be any gene that activates transcription of the target gene suppressor either by direct binding or by causing an effect or a cascade of effects that lead to transcription activation. One example of the transcription-activating gene is a site-specific DNA recombinase, i.e. the Cre-loxP system or the Flp/frt system. Recombination of Cre or Flp at the specific loxP or frt sites, respectively, can bring the target gene suppressor next to its promoter sequence, by deleting the intervening DNA fragment, and thereby leading to transcription. The intervening DNA fragment may comprise coding sequence for a marker gene and its accompanying promoter. Elimination of the marker gene expression is an indication of the Cre or Flp mediated recombination event. The transcription-activating gene can also be a transposon. A transposon can insert into a gene at specific sites and can be excised from the gene into which it is inserted. In the present invention involving a target gene suppressor, the excision of the transposon restores the gene suppressor to its original state and activates the transcription of the target gene suppressor from its promoter.

In its more particular aspect, the invention contemplates the repressible promoter, for either the transcription-activating gene or the target gene suppressor or both, being spatial-specific, i.e. transcribed only in certain locus of the cell DNA. Alternatively, the transcription can be tissue specific, i.e. transcribed only in certain cell type such as liver cells.

It is also important to be able to control the onset of the expression of the suppressor gene in gene knockdown animal models. If the target gene is important to embryonic development, suppression of the target gene may lead to a lethal impact during embryogenesis. Alternatively, if the transgene product is highly toxic to the organism, it can be detrimental to the developing embryo. These factors can prevent the abnormal phenotype from reaching maturity which precludes study on the full scope of the gene function. Thus, in the instant invention, the target gene suppressor in the animal model is not only spatial specific and but also temporally controlled.

In a further embodiment of the invention, temporal control is carried out via transcriptional repressors binding to the repressible promoter. The transcriptional repressor is responsive to an exogenous molecule, an induction agent, which can be delivered at any given time, accounting for the temporal control. Upon delivery of the induction agent, the transcriptional repressor will bind to the induction agent instead of the repressible promoter, leading to transcription from the promoter. Upon removal of the induction agent, the transcriptional repressor will bind to the repressible promoter leading to inhibition of transcription from the promoter. One example of such an induction system is the tetracycline-on system. Under this system, tetracycline repressor is the transcriptional repressor that binds to the tetracycline responsive element on the tissue-specific promoter but will come off and bind to tetracycline in the presence of tetracycline, consequently, the transcription-activating gene will be transcribed in a specific type of cells. Any tetracycline analog can also be employed with modified responsive element and transcriptional repressor suitable for binding by the tetracycline analog. Similarly, any other transcriptional repressor/induction agent system can also be used, for example various steroidal compounds, divalent cations (including Cu.sup.+2 and Zn.sup.+2), galactose, IPTG (isopropyl .beta.-D thiogalactoside), as well as other naturally occurring and synthetic inducing agents. In addition, the transcriptional repressor can be under the control of one or more induction systems which may be the same or different.

In yet another embodiment, one or both of the first and second repressible promoters are spatial-specific and the transcriptional repressor is also temporally controlled. In this manner, the target gene suppressor is under a spatiotemporal control in that it is only transcribed in certain cell type and the transcription can be turned on or off at any given time by introducing or removing the induction agent. Thus, the cell-specific gene knockdown system generated is reversible, and the transgenic animal created utilizing this method expresses a reversible phenotype.

In a more particular embodiment, the target gene suppressor employed to suppress gene expression is interference RNA. Any interference RNA for a selected gene can be used. As an example, siRNA for the ABCA1 gene is applied to prevent translation of the ABCA1 mRNA.

In another aspect of the present invention, the transcription-activating gene regulating the gene suppressor is a transposon element. In yet another embodiment of the reversible gene knockdown system, the transcription-activating gene is a site-specific DNA recombinase, e.g. a Cre or Flp recombinase.

In a further embodiment of the invention, the transcriptional repressor for the transcription-activating gene and or gene suppressor is regulated by an induction agent, such as tetracycline, doxycycline, or other tetracycline analogs.

In an alternative aspect of the invention, the transcription-activating gene for the target gene suppressor is a site specific DNA recombinase and the transcriptional repressor is an induction agent regulated repressor. The induction agent may be tetracycline, doxycycline or other tetracycline analogs. The target gene suppressor may be siRNA. Alternatively, the DNA recombinase may be Cre specifically. Furthermore, in another embodiment, siRNA, as the target gene suppressor, is regulated by Cre recombinase.

In another embodiment, the transcription-activating gene, i.e. site-specific DNA recombinase, is spatial specific and the transcriptional repressor is temporally controlled via tetracycline regulated repressor or tetracycline analog regulated repressors. The target gene suppressor may be siRNA. The site-specific DNA recombinase utilized may be Cre. Alternatively, the target gene suppressor, siRNA, is regulated by Cre recombinase.

The invention also provides another method for producing a reversible gene knockdown system. A nucleic acid molecule comprising a coding sequence for at least one target gene suppressor is introduced into a nonhuman mammal. The target gene suppressor is regulated by a first repressible promoter controlled by a transcription-activating gene. The first repressible promoter is also under the control of a first transcriptional repressor. The transcription-activating gene is regulated by a second repressible promoter under the control of a second transcriptional repressor. The first and second transcriptional repressors may be the same or different. In addition, there can be one or more first transcriptional repressors and/or one or more second transcriptional repressors.

The invention further contemplates a method for producing a reversible gene knockdown system via mating two transgenic animals. The method comprises introducing into a first nonhuman mammal a nucleic acid molecule comprising a coding sequence for at least one target gene suppressor. The expression of the at least one target gene suppressor is regulated by a first repressible promoter. The first repressible promoter is controlled by at least one transcription-activating gene. The first repressible promoter is also under the control of at least one transcriptional repressor. The method further comprises introducing into a second nonhuman mammal a nucleic acid molecule comprising a coding sequence for at least one transcription-activating gene, a second repressible promoter, and the at least one transcriptional repressor, wherein expression of the at least one transcription-activating gene is regulated by the second repressible promoter under the control of the at least one transcriptional repressor. Subsequently, the first and second nonhuman mammals are mated to form an offspring mammal having nucleic acid molecules from both parents. Said nucleic acid molecules should comprise the coding sequence for at least one target gene suppressor regulated by a first repressible promoter which is controlled by at least one transcription-activating gene. The first repressible promoter is also under the control of at least one transcriptional repressor. It also comprises the coding sequence for at least one transcription-activating gene, a second repressible promoter, and the at least one transcriptional repressor, wherein expression of the at least one transcription-activating gene is regulated by the second repressible promoter under the control of the at least one transcriptional repressor.

In a further embodiment, the invention provides a method of producing a reversible gene knockdown system that comprises introducing into a first nonhuman mammal a nucleic acid comprising a coding sequence for at least one target gene suppressor regulated by a first repressible promoter which is under the control of both a transcription-activating gene and a first transcriptional repressor. The method further comprises introducing into a second nonhuman mammal a nucleic acid comprising a coding sequence for at least one transcription-activating gene, a second repressible promoter, and a second transcriptional repressor, wherein expression of the at least one transcription-activating gene is regulated by the second repressible promoter under the control of the second transcriptional repressor. Then the first and second nonhuman mammals are mated to form an offspring mammal having the nucleic acid molecules from both the first and second nonhuman mammals, which nucleic acid molecules have the first and second transcriptional repressors. The first and second transcriptional repressors may be the same or different. Furthermore, there can be one or more first transcriptional repressors and/or one or more second transcriptional repressors.

The method for producing an inducible gene knockdown animal is the same as the method for producing a reversible gene knockdown system except the promoter for the target gene suppressor does not contain a binding site for the transcriptional repressor. Thus, the target gene suppressor is not under the control of any transcriptional repressor. Once the target gene suppressor is turned on by the transcription-activating gene, it is always on and can not be repressed. Consequently, the gene knockdown is inducbile but not reversible.

The inducible gene knockdown animal can be created by introducing into the nonhuman mammal nucleic acid molecules comprising a coding sequence for at least one target gene suppressor wherein expression of the at least one target gene suppressor is regulated by a promoter controlled by at least one transcription-activating gene. The at least one transcription-activating gene is regulated by a repressible promoter under the control of at least one transcriptional repressor.

In yet another embodiment, the inducible gene knockdown animal can be created by introducing into a first nonhuman mammal a nucleic acid comprising a coding sequence for at least one target gene suppressor wherein expression of the at least one target gene suppressor is regulated by at least one promoter under the control of at least one transcription-activating gene. The method further comprises introducing into a second nonhuman mammal a nucleic acid comprising a coding sequence for the at least one transcription-activating gene, at least one repressible promoter, and at least one transcriptional repressor, wherein the expression of the at least one transcription-activating gene is regulated by the at least one repressible promoter under the control of the at least one transcriptional repressor. The first and second nonhuman mammals are mated such that the offspring mammal have nucleic acid molecules from both the first and the second nonhuman mammal.

In a particular embodiment of the invention, the reversible gene knockdown animal was created through mating one transgenic mouse, which carried the siRNA/LoxP plasmid, with another mouse, which carried the expression of site specific DNA recombinase, Cre, under the control of a tissue-specific promoter and tetracycline repressor. The vectors are designed to enable expression of siRNA under the control of U6 promoter after the recombination of the two LoxP sites by Cre recombinase. The mating produces an offspring animal that carries the transgenes from both parents. The regulation of RNA interference in the offspring of transgenic mouse was strictly through the tetracycline-on system. The tissue specific Cre recombinase is produced upon induction by tetracycline. Cre recombinase in turn mediated the recombination of two LoxP sites that lead to the switching on of the siRNA transcription cassette in the same cell. In this embodiment, ABCA1 is used as a target gene of the study. The recombination mediated by Cre recombinase leads to transcription of ABCA1 siRNA which efficiently blocks the ABCA1 expression in the specific tissue.

The invention provides a method for producing a reversible gene knockdown system comprises introducing into a nonhuman mammal a nucleic acid comprising a coding sequence for at least one target gene suppressor regulated by a repressible promoter which is under the control of at least one transcription-activating gene and at least one transcriptional repressor. The transcription-activating gene is regulated by an inducible promoter, which can be a stress promoter. The stress promoter can be a heat shock promoter.

The invention also contemplates a method for producing an inducible gene knockdown system by introducing into a nonhuman mammal a nucleic acid encoding a target gene suppressor regulated by a repressible promoter. The repressible promoter is under the control of a transcription-activating gene, and the transcription-activating gene is in turn regulated by an inducible promoter. The inducible promoter can be stress promoter, such as a heat shock promoter.

The nucleic acid molecule introduced into the nonhuman mammals may be linear or circular such as plasmids. The nucleic acid molecule can comprise terminal repeats or any other sequences for ease of integration into the host genome. The recombinant DNA construct may be introduced into the non-human mammal by various methods as described below.

The transgene construct can be introduced into a single stage embryo. The zygote, the formation of a diploid cell which is capable of developing into a functioning organism whethre it be euploid or an euploid zygote, is the best target for micro-injection. In the mouse, the male pronucleus reaches the size of approximately 20 micrometers in diameter which allows reproducible injection of 1-2 μl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the exogenous genetic material into the embryo may be accomplished by any means known in the art, such as for example, microinjection, electroporation, or lipofection, as long as it is not destructive to the cell nuclear membrane or other existing cellular or genetic structures. Following introduction of the transgene nucleotide sequence into the embryo, the embryo may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockdown; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will be a DNA sequence which results in the production of a polynucleotides or polypeptides that suppress gene function. Further, in such embodiments the sequence will be attached to a transcriptional control element, e.g., a promoter, which preferably allows the expression of the transgene product in a specific type of cell and or at certain predetermined time or stage, a transcription-activating gene and or a transcriptional repressor.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review, see Jaenisch, R. (1988) Science 240:1468-1474.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for expression of the transgene by methods described above. For example, screening may be carried out with a green fluorescent protein marker, Southern blots and/or PCR analysis for presence of the transgene, Northern blots to probe mRNA transcribed from the transgene, and Western blots using antibody to probe presence of protein translated from the transgene.

Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Yet other methods of introducing the transgene material into an animal make knockdown or disrupt transgenic animals are also generally known. See, for example, Manipulating the Mouse Embryo, (Cold Spring Harbor Laboratory Press, Cold Spring Harbor, N.Y., 1986).

Transgenic Animal

The instant invention also includes the nonhuman mammals, carrying the transgene with the target gene suppressor and its various regulating sequences described above. The transgenic animal can be made through introduction of transgenic material according to the forementioned methods.

In one embodiment, the transgenic animal is the founder animal, the first original transgenic animal, and carries reversible gene knockdown transgene in one nucleic acid molecule. In another embodiment, the transgenic animal is an offspring derived from mating two transgenic founder animals with each carrying a different transgene. Thus, the transgenic offspring animal carries the transgenes from both parents. The complement of the two transgenes constitutes the reversible gene knockdown system.

In another particular embodiment, there is the transgenic founder animal carrying the inducible gene knock dwon transgene in one molecule. Alternatively, there is the nonhuman mammal which is an offspring derived from mating two transgenic founder animals, each carrying a different transgene. The offspring carries two trangenes from the parents and the complement forms the inducible gene knockdown system.

The present invention is also directed to a method to reversibly knockdown gene expression in a nonhuman mammal by first providing for a transgenic animal as described above and then administer an exogenous molecule, i.e. an induction agent, to induce expression inhibition. Upon removal of the induction agent, gene expression is restored. Thus, gene knockdown in the nonhuman mammal is reversible.

Recombinant DNA Construct

The recombinant DNA construct generally comprises, in the order of 5′ to 3′, the promoter sequence and the target gene suppressor. Depending on the transcription-activating gene used, there may be intervening sequences between the promoter and the target gene suppressor. Furthermore, the promoter sequence may be bound by a transcriptional repressor. Two levels of regulating the target gene suppressor, by the transcription-activating gene and the transcriptional repressor, enables spatial and temporal control of the gene suppressor.

An embodiment of the reversible gene knockdown system comprises a recombinant DNA construct comprising a nucleic acid comprising a coding sequence for at least one target gene suppressor which is regulated by a first repressible promoter. The first repressible promoter is controlled by at least one transcription-activating gene. The first repressible promoter is also under the control of at least one transcriptional repressor. Expression of the at least one transcription-activating gene is regulated by a second repressible promoter under the control of the at least one transcriptional repressor.

In yet another embodiment of the reversible gene knockdown system, there is a recombinant DNA construct comprising a nucleic acid comprising a coding sequence for at least one target gene suppressor and a first repressible promoter for regulating the expression of the at least one target gene suppressor. Regulation of the at least one target gene suppressor by the first repressible promoter is controlled by at least one transcription-activating gene. The first repressible promoter is also under the control of a first transcriptional repressor. Expression of the at least one transcription-activating gene is regulated by a second repressible promoter under the control of a second transcriptional repressor.

In another embodiment, in an inducible gene knockdown system, there is a recombinant DNA construct comprising a nucleic acid comprising a coding sequence for at least one target gene suppressor wherein expression of the at least one target gene suppressor is regulated by a promoter controlled by at least one transcription-activating gene and wherein the expression of the at least one transcription-activating gene is regulated by a repressible promoter under the control of at least one transcriptional repressor.

Those skilled in the art are well able to construct vectors and design protocols for recombinant gene expression. For further details see, for example, Molecular Cloning: a Laboratory Manual: 2nd edition, Sambrook et al, 1989, Cold Spring Harbor Laboratory Press.

Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second edition, Ausubel et al. eds., John Wiley & Sons, 1992.

Transfected Cells

The present invention also provides cultured cells and embryonic stem cells, stably transformed or transfected with the above-described DNA recombinant construct by means known in the art. Furthermore, the transfected cell may be obtained from an animal transfected with the recombinant DNA construct.

The invention contemplates isolated cells or a cell culture derived from a gene knockdown transgenic animal of the present invention. The cells can be obtained directly from the animal, from a descendent animal, or can be a progeny of a primary culture of one or more cells of the animal. The isolated cell can be in the form of a single cell or cell line, or a composition of mixed cells. The cell can be obtained from an animal which is the descendant of a transgenic animal of this invention, such as by a cross with another animal having either the same or a different genetic background. Thus the isolated cell can be homozygous or heterozygous for the transgene.

The transgene construct to be inserted into the cell may be set in the linear form. Therefore, if the transgene construct has been inserted into a vector, linearization is accomplished by digesting the DNA with a suitable restriction endonuclease selected to cut only within the vector sequence and not within the transgene construct sequence.

For insertion into ES cells, the transgene construct is added to the ES cells under appropriate conditions for the insertion method chosen, as is known to the skilled artisan, such as electroporation. The cells are then screened for the presence of the transgene construct. Where more than one construct is to be introduced into the ES cell, each transgene construct can be introduced simultaneously or one at a time.

Both the foregoing general description and the following detailed description are exemplary and explanatory only and are not restrictive of the invention, as claimed. Moreover, the invention is not limited to the particular embodiment described, as such may, of course, vary. Further, the terminology used to describe particular embodiments is not intended to be limiting, since the scope of the present invention will be limited only by its claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The following examples further illustrate the invention. They are merely illustrative of the invention and disclose various beneficial properties of certain embodiments of the invention. The following examples should not be construed as limiting the invention.

### EXAMPLES

The present invention is further illustrated by the following examples which should not be construed as limiting in any way. The contents of all cited references, including literature references, as cited throughout this application are hereby expressly incorporated by reference.

The practice of the present invention will employ, unless otherwise indicated, conventional techniques of cell biology, cell culture, molecular biology, microbiology and recombinant DNA, which are within the skill of the art. Such techniques are explained fully in the literature.

The following examples illustrates the preparation of transgenic mice expressing an inducible and a reversible gene-knockdown system using ABCA1 (ATP-binding cassette subfamily A member 1) as the target gene under the control of interference RNA targeting ABCA1 (ABCA1 siRNA), Cre recombinase, and tetracycline repressor (rtTA).

### Example 1

### Plasmid Construction

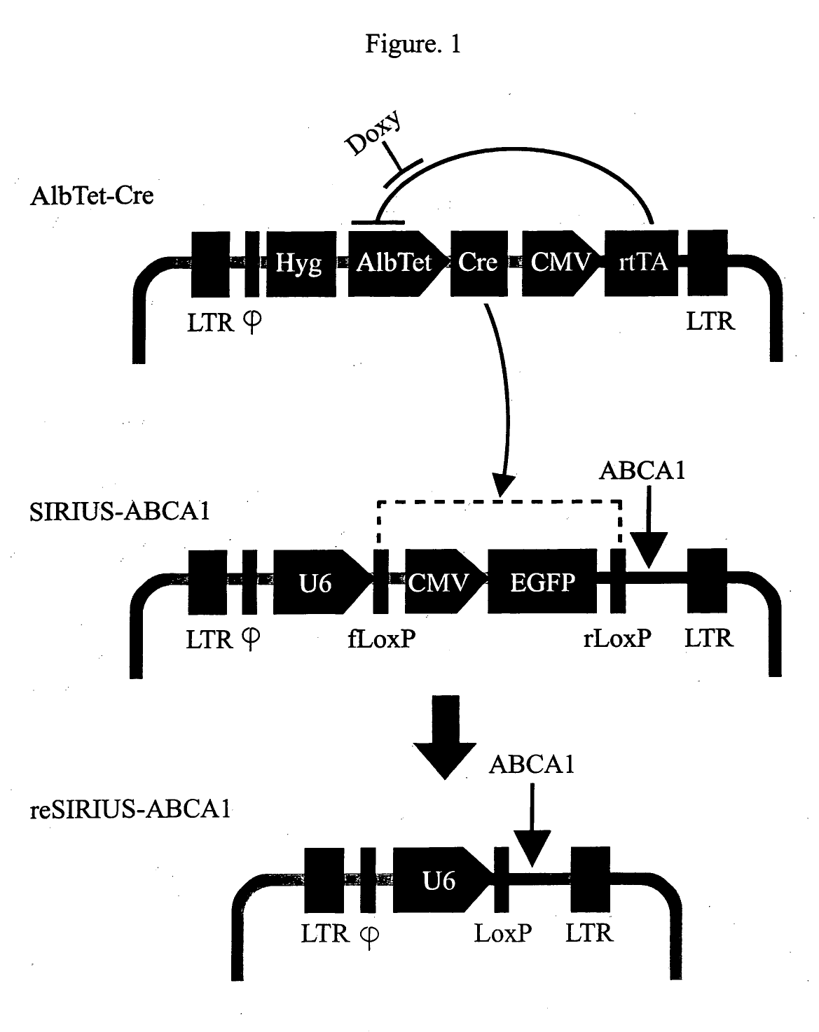

To create a transgenic mouse, the first step is to prepare the vectors carrying the transgene. For this purpose, two vectors were constructed, SIRIUS-ABCA1 and Alb-Tet Cre. Recombination of the two vectors yielded reSIRIUS-ABCA1, see FIG. 1. (Abbreviations for FIG. 1 are LTR, Long Terminal Repeat; TRE, Tet-responsive element; Neo, Neomycin resistance gene; Hyg, Hygomycin resistance gene; ABCA1, ATP-Binding Cassette subfamily A member 1)

The SIRIUS-ABCA1 vector expressed an EGFP (Enhanced Green Fluorescent Protein) cDNA under the control of a CMV (cytomegalovirus) promoter, also referred to collectively as the “EGFP cluster”. The EGFP cluster is flanked by two loxP sites, i.e. rloxP and floxp, located down and upstream of the EGFP cluster respectively. The ABCA1 siRNA is located down-streatm of rloxP, and the controlling promoter, U6 RNA polymerase III promoter, is up-stream from floxP. The vector expressed the hairpin oligo of ABCA1 siRNA under the control of the mouse U6 promoter after recombining two LoxP sites by Cre recombinase. This vector was constructed according to the steps described below.

First, an U6 promoter, which contained a Mlu I cloning site at the 3′ end was inserted into the Bc/I site of the pLEGFP-C1 vector (BD Biosciences). Second, two LoxP oligos were inserted at the Xho I, Apa I, and Mlu I restriction sites down- and up-stream from the EGFP cluster. The sequence of the LoxP is 5′-ATA ACT TCG TAT AGC ATA CAT TAT ACG AAG TTA T-3′. Third, an oligonucleotide encoding a hairpin siRNA against ABCA1, being an inverted repeat separated by a 9-nt (TTC AAG AGA) spacer, was inserted downstream of the rLoxP using the Bam HI and Not I cloning sites. The sequence of the oligonucleotide encoding the ABCA1 siRNA hairpin is 5′-GAA CCT CAC TTT CAG AAG ATT CAA GAG ATC TTC TGA AAG TGA GG-3′.

The AlbTet-Cre vector contained a modified liver-specific human albumin promoter with TRE (Tetracycline Responsive Element), Cre recombinase and tetracycline repressor—rtTA. The vector expressed the Cre recombinase under the control of the albumin promoter and doxycycline, an analog of tetracycline. This vector was constructed according to the following steps.

First, full-length cDNA of the Cre gene was inserted into Bgl II site of the pLHCX vector (BD Biosciences). Next, Mlu I and Apa I sites were created upstream of the Cre gene, and then a modified albumin promoter was inserted into Mlu I and Apa I sites of the pLHCX vector. Then, a fragment encoding TRE was created next to and downstream of albumin promoter. Finally, the tetracycline repressor, rtTA, was inserted at the 3′ end of the CMV promoter using the Hind III and Hpa I sites. The sequence of TRE is 5′-CTC CCT ATC AGT TGA TAG AGA AA-3′.

The recombinant reSIRIUS-ABCA1 plasmid, when created in vivo from these plasmids, should have two distinct features; it will be smaller in size due to LoxP recombination caused by the Cre recombinase and it will have lost fluorescence for a similar reason. Cre recombinase is produced under liver albumin promoter control through doxycycline induction.

### Example 2

### In Vivo Recombination Assay in HepG2 Cells

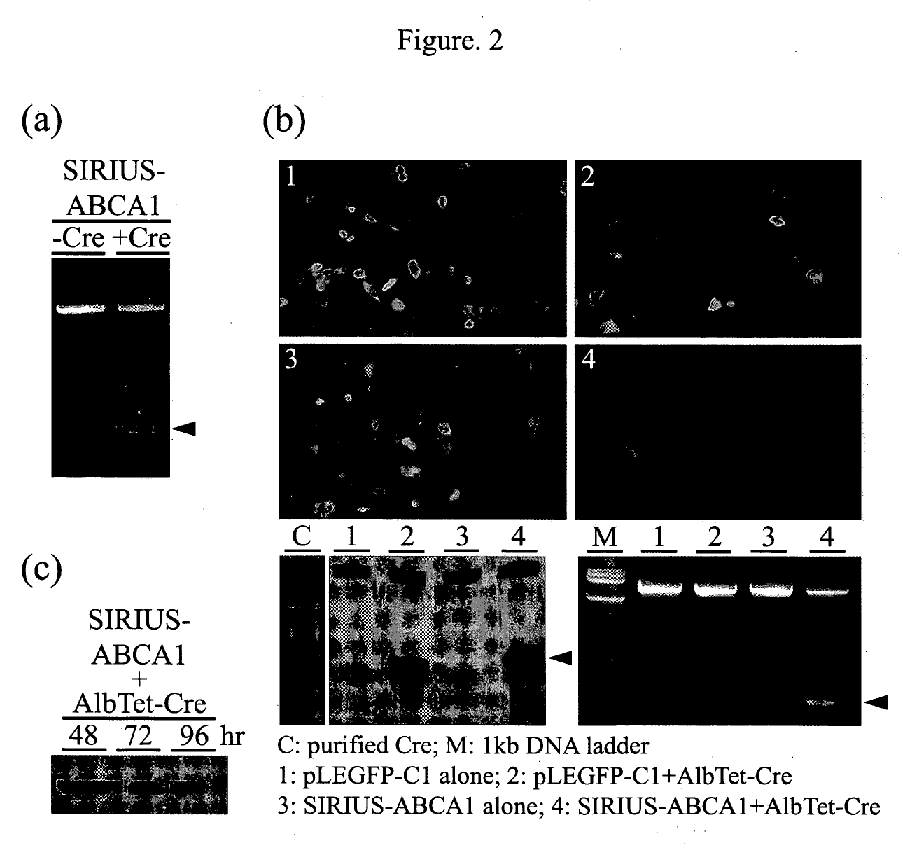

In order to determine the effect of the Cre recombinase on SIRIUS-ABCA1, an in vivo recombination assay was first carried out and the results are shown in FIG. 2.

Specifically, liver cells, HepG2 cells, were transfected with SIRIUS-ABCA1 plasmid 24 hour after passage using the manufacture's protocol of siPORT™ amine transfection agent (Ambion Inc.). The transfected cells were treated with purchased Cre recombinase (Invitrogen) and the repression of SIRIUS-ABCA1 was assayed by RT-PCR using specific primers (p1581: 5′-TTA TCC AGC CCT CAC TCC TTC T-3′ and p4214: 5′-CAT TAA GGG ATC AGT TAT CTA G-3′) for reverse transcription and specific primers for ABCA1 (ABCA1F: 5′-CCT TGG GTT CAG GGG ATT AT-3′; ABCA1R: 5′-GGC TTC AGG ATG TCC ATG TT-3′) for PCR analysis.

PCR analysis of the recombined SIRIUS-ABCA1 after treatment (for 2 hr at 37° C.) with purified Cre recombinase (Invitrogen) using specific primers shows a shorter fragment. The fragment was reduced to less than 0.5 kb after Cre recombinase treatment where the CMV promoter and EGFP were knocked out, FIG. 2(a). Without Cre recombinase treatment, where LoxP recombination did not happen, a 2.6 kb PCR fragment was detected, FIG. 2(a). Both PCR fragments were close to estimated sizes of the original vector and the Cre recombinase treated vector.

Then HepG2 cells were co-transfected with SIRIUS-ABCA1 and AlbTet-Cre with tetracycline in culture medium. These SIRIUS-ABCA1/AlbTet-Cre co-transfected cells displayed down-regulation of GFP (Green Fluorescent Protein) expression, see FIG. 2(b). Cells co-transfected with pLEGFP-C1 in which LoxP was not incorporated, however, did not show any fluorescence reduction. The disappearance of fluorescence was attributed to the elimination of the CMV and EGFP fragments by the Cre recombinase mediated LoxP recombination. Fluorescence pictures of HepG2 cells transfected either with pLEGFP-C1 alone or SIRIUS-ABCA1 alone, or co-transfected with pLEGFP-C1 and AlbTet-Cre or SIRIUS-ABCA1 and AlbTet-Cre, respectively were examined, FIG. 2(b). Under the fluorescent microscope, the fluorescence level shown by cells transfected by pLEGFP-C1 alone, SIRIUS-ABCA1 alone, and pLEGFP-C1/AlbTetCre were about the same, i.e. fluorescence present throughout the specimen. However, there was only a single spot showing fluorescence in the specimen of cells transfected with SIRIUS-ABCA1/AlbTetCre. Photography displays significant down-regulation of GFP expression in SIRIUS-ABCA1/AlbTet-Cre transfected cells.

Western blot analysis of Cre recombinase and genomic PCR analysis was then carried out with respect to the co-transfected cells in the tetracycline medium to demonstrate the recombination process, see FIG. 2(b). Cellular lysates (1: pLEGFP-C1 alone, 2: pLEGFP-C1/AlbTet-Cre, 3: SIRIUS-ABCA1 alone, and 4: SIRIUS-ABCA1/AlbTet-Cre) were prepared 72 hr after transfection with different combinations of vectors. Western blot analysis demonstrated that there was significant expression of the Cre recombinase only in cells transfected by pLEGFP-C1/AlbTet-Cre and SIRIUS-ABCA1/AlbTet-Cre using Cre polyclonal antibody (Novagen), see FIG. 2(b) Left Panel lane 2 and lane 4. Cells transfected with pLEGFP-C1 alone or SIRIUS-ABCA1 alone did not produce Cre recombinase, see FIG. 2(b) Left Panel lane 1 and lane 3. The commercial Cre recombinase (Invitrogen) was analyzed as a positive control (con). Genomic-PCR analysis showed that the recombination happened in HepG2 cells when transfected with SIRIUS-ABCA1/AlbTet-Cre, resulting in shorter DNA fragments. The expressed Cre recombinase displayed its effect on LoxP recombination as shown by the RT-PCR results, see FIG. 2(b) Right Panel, which are similar to results shown in FIG. 2(a).

Subsequently, to obtain evidence that expression of ABCA1 was decreased in these cells, Western blotting using the ABCA1 antibody purchased from Santa Cru, Inc. (cat no. sc-5491) was employed with SIRIUS-ABCA1/AlbTet-Cre transfected cell lysates, and the results are shown in FIG. 2(c). This clearly demonstrates that ABCA1 was progressively reduced based on the time period (48, 72 and 96 hr) after infection. The ABCA1 quantity dropped to approximately 30 of the original level 96 hr after transfection.

### Example 3

### Recoverable System

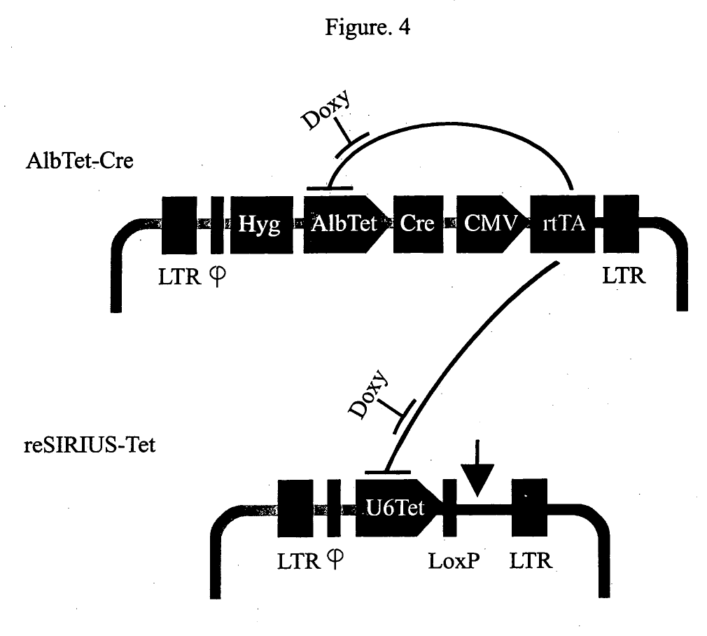

A recoverable system is constructed based on the SIRIUS-ABCA1 and AlbTet-Cre constructs described in Example 1. THE SIRIUS-ABCA1 vector is inserted with a TRE (tetracycline responsive element) fragment, depicted in the figure as “Tet”, which is next to and down-stream from the U6 promoter and under the control of rtTA, such as that found on the AlbTet-Cre vector: creating the SIRIUS-Tet vector, see FIG. 4. Upon removal of doxycycline, rtTA repressor will bind to both the albumin and the U6 promoters. Binding to the U6 promoter blocks the siRNA expression. The blocking of the siRNA expression further lead to target gene re-expression.

### Example 4

### Preparation of Transgenic Animals

Having proved in HepG2 cells that upon induction by doxycycline, AlbTet-Cre does produce Cre recombinase, which in turn recombines the two sites of LoxP in SIRIUS-ABCA1 leading to the expression of functional ABCA1 siRNA. The SIRIUS-ABCA1 and AlbTet-Cre plasmids were prepared by CsCl gradient and injected separately into fertilized mouse oocytes to generate transgenic mice.

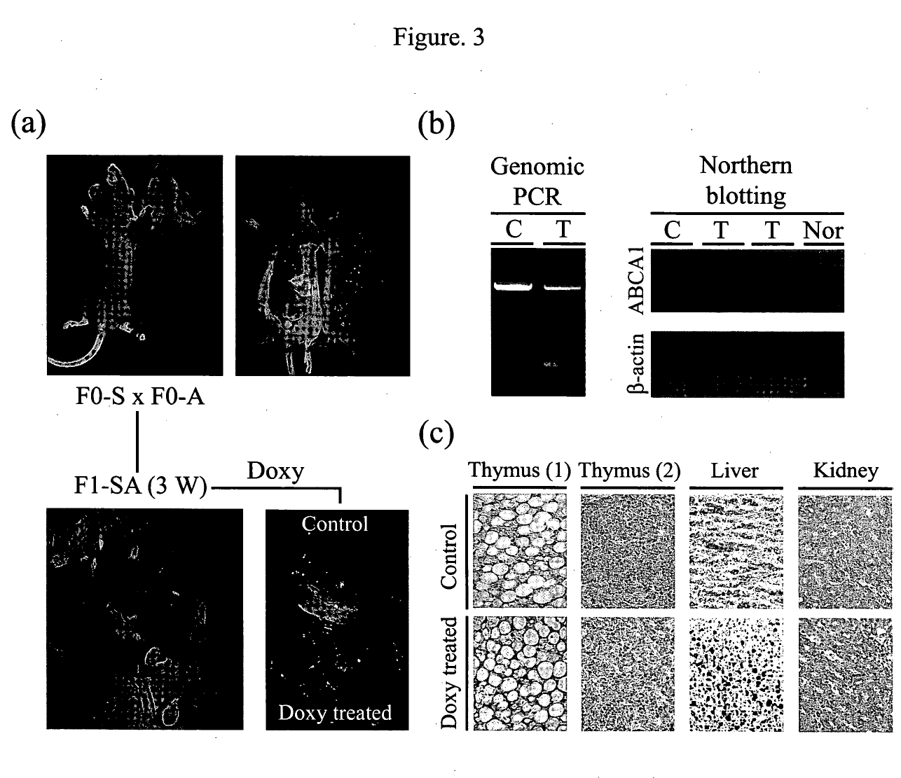

In general, two separate lines of transgenic animals carrying AlbTet-Cre (F0-A) and SIRIUS-ABCA1 (F0-S) plasmid, respectively, were created. The F0-S animals were examined under a fluorescent microscope to visualize expression of EGFP, FIG. 3(a). The two separate lines of transgenic mice were mated to generate offspring (F1-SA) that carry both transgenes in a single mouse. F1-SA animals were also examined under a fluorescent anatomy-microscope to visualize the expression pattern of EGFP, see FIG. 3(a). Liver tissues from F1-SA mice were sectioned and visualized after treatment of doxycycline, FIG. 3(c). Genomic-PCR analysis of the recombination of SIRIUS-ABCA1 in livers of doxycycline treated and untreated mice was carried out. Doxycycline treated F1-SA mice exhibited a less than 0.5 kb PCR product, same as that which is shown in HepG2 cells in FIG. 2(b), see FIG. 3(b). Also, Northern blot analysis of ABCA1 expression was performed with untreated (control, C) and treated (treated, T) F1-SA mice. 10 μg RNA was separated on gel and transferred to a membrane. A specific-ABCA1 probe was used to detect the expression of ABCA1 in the Northern blot. Beta-actin was used as internal control of quantification, see FIG. 3(b).

More specifically, in creating the transgenic mice, the genes to be incorporated into the mouse genome were prepared. The DNAs of both transgene vectors were cut with specific restriction enzymes, using Sca I for SIRIUS-ABCA1 and Afl III for AlbTet-Cre, after purification by CsCl gradient centrifugation. The DNA was diluted to 5 ng/ul in sterile MilliQ water for injection into embryos at pronucleus stage.

Then, one-cell embryos of adult ICR mice (>4 weeks old) were collected after superovulation as described (Choo K. B. et al., (2001) Mol. Reprod. Dev. 59: 249-255). Eluted embryos were maintained in DPBS medium under an atmosphere of 5% CO2 at 37.

The prepared DNA fragments were then injected into the embryo at the pronucleus stage by pronucleus injection.

These zygotes were re-implanted into pseudo-pregnant foster mothers, and the offspring were screened for presence of the transgene by Southern blotting and PCR analysis, respectively. Genomic DNA was isolated from the tail blood of the transgenic animals by proteinase K digestion followed by phenol extraction and ethanol precipitation. PCR analyses were used to identify transgenic mice using the specific primers (p1581 and p4214 primers for SIRIUS-ABCA1 and the CreF and CreD primers for AlbTet-Cre). The primer sequences of CreF and CreD were 5′-ATG GCA CCC AAG AAG AAG AGG A-3′ and 5′-CTA ATC GCC ATC TTG CAG CAG G-3′, respectively. Integration of the SIRIUS-ABCA1 construct into the genome of potential founders was assessed, and these mice were named F0-S and F0-A for the incorporation of SIRIUS-ABCA1 and AlbTet-Cre respectively, see FIG. 3(a). Three founders (F0-S) successfully passed the transgene through the germ line. GFP expression in the F0-S animals and their organs can all be visualized using fluorescent anatomy-microscopy, see FIG. 3(a) which shows fluorescent F0-S animals and fluorescent organs within the F0-S animals. No fluorescence was emitted by the F0-A animals. Also, the presence of the AlbTet-Cre construct had been confirmed and two founders (F0-A) successfully passed the transgene to their offsprings. All transgenic mice of founders developed and bred normally.

Offspring, F1-SA, of the F0-S and F1SA animals were produced through spontaneous mating soon after animals reached puberty, FIG. 3(a). It was shown that the F1-SA animals simultaneously carried intact SIRUS-ABCA1 and AlbTet-Cre genes as proved by GFP expression under a fluorescent anatomy-microscope as described earlier for the initial F0-S, FIG. 3(a). In this study, four F1-SA mice successfully carried two transgenes and that genomic DNA of F1-SA was detected by PCR and microscopy assays.

Then, F1-SA transgenic animals (one week old) were supplied with doxycycline hydrochloride for four days via breast milk (2 mg/ml doxycycline/ml). To enhance the induction, 4 mg/ml doxycycline in 0.5 ml of normal saline was injected intraperitoneally twice at intervals of 24 hr.

Doxycycline treatment of F1-SA mice abolished GFP expression, demonstrating excision of the GFP gene by Cre-mediated recombination. Tissue-specific AlbTet control of Cre is active, as shown by the differential expression of Cre between the liver and gall bladder (FIG. 3(a).

Besides the fluorescence visualization, Cre-induced LoxP recombination in the F1-SA offspring can also be proved by a genomic PCR study of SIRUS-ABCA1 using specific primers. The 2.6 kb segment of DNA was reduced to less than 0.5 kb after doxycycline treatment, see FIG. 3(b), Left Panel, and the result is the same as in the Cre treatment studies of HepG2 cells described earlier in FIG. 2(a) and FIG. 2(b) Right Panel.

A Northern Blot analysis was also carried out, and the actual reduction of ABCA1 RNA in doxycycline treated F1-SA mice can be seen in the Northern blot analysis, FIG. 3(b), Right Panel. Ten micro-grams RNA of liver was separated on gel and transferred to a membrane. A specific-ABCA1 oligo-probe (ACA CCT GGA GAG AAG CTT TCA ACG AGA CTA ACC) was end-labeled and used to detect the expression of ABCA1 in Northern blot. ABCA1 is reduced to an almost non-detectable level 96 hr after doxycycline treatment in the treated F1-SA transgenic animals (treated, T) whereas untreated F1-SA transgenic animals (control, C) as well as normal (non-transgenic) mice exhibited a high level of ABCA1, FIG. 3(c).

Therefore, in sum, the method of producing these transgenic mice was via mating of one mouse, which carried the siRNA/LoxP plasmid, with another mouse, which carried expression of Cre recombinase in specific tissues. The regulation of the RNAi in the offspring of transgenic mice was strictly through the tetracycline-on system. Upon induction by tetracycline, the tissue specific Cre recombinase were produced and in turn mediated the recombination of two LoxP sites that lead to the switching on of the siRNA transcription cassette in the same cell.

### Example 5

### Proof of Suppression of ABCA1 at Tissue Level

The ultimate proof of a successful transgenic model in this case, of course, is the deposition of cholesterol in liver due to the inhibition of ABCA1 in F1-SA transgenic mice. Cholesterol was detected using the Oil Red O staining method and its presence in liver specimen was compared between treated and untreated mice.

Liver tissues derived from the untreated (control, C) F1-SA transgenic mice and treated (treated, T) F1-SA mice were used. Tissues taken for lipid analysis were frozen in O.C.T (optimal cutting temperature) medium. Frozen sections (5 μm in thickness) were stained with Oil Red O for cholesterol display and were counterstained with hematoxylin. Liver specimens clearly exhibit higher level of cholesteryl esters staining after doxycycline treatment (400×), see FIG. 3.

Lipid staining in liver of a doxycycline treated F1-SA mouse shows an overwhelming red color whereas the color is almost non-detectable in the liver of the untreated control ones, see FIG. 3(c). These results clearly show that this approach has created a stable, inheritable and inducible transgenic animal model with the hallmark of high cholesterol content in liver by using this new transgenic system.

### REFERENCES

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

1-197. (canceled)

198. A nucleic acid construct comprising at least one target gene suppressor under the control of at least one first promoter under the control of at least one transcription-activating gene under the control of at least one second promoter.

199. The nucleic acid construct of claim 198, wherein the at least one second promoter is a repressible promoter under the control of at least one transcriptional repressor.

200. The nucleic acid construct of claim 199, wherein the at least one first promoter is another repressible promoter also under the control of the at least one transcriptional repressor.

201. The nucleic acid construct of claim 200, wherein the at least one transcriptional repressor controlling the at least one first promoter and controlling the at least one second promoter are different transcriptional repressors.

202. The nucleic acid construct of claim 200, wherein the at least one first promoter is tissue-specific and the at least one target gene suppressor is siRNA.

203. The nucleic acid construct of claim 202, wherein the tissue specific repressible promoter is liver-specific.

204. The nucleic acid construct of claim 202, wherein the siRNA is ABCA1 siRNA.

205. The nucleic acid construct of claim 199, wherein the at least one transcription-activating gene is a transposon element.

206. The nucleic acid construct of claim 199, wherein the at least one transcription-activating gene is a site-specific DNA recombinase.

207. The nucleic acid construct of claim 206, wherein the site-specific DNA recombinase is selected from Cre or Flp.

208. The nucleic acid construct of claim 199, wherein the transcriptional repressor is an induction agent regulated repressor.

209. The nucleic acid construct of claim 208, wherein the induction agent regulated repressor is selected from a tetracycline regulated repressor or a tetracycline analog regulated repressor.

210. The nucleic acid construct of claim 199, wherein the transcription-activating gene is a site-specific DNA recombinase and the transcriptional repressor is an induction agent regulated repressor.

211. The nucleic acid construct of claim 210, wherein the at least one target gene suppressor is siRNA.

212. The nucleic acid construct of claim 211, wherein the site-specific DNA recombinase is Cre.

213. The nucleic acid construct of claim 212, wherein the induction agent that regulates the transcriptional repressor is selected from tetracycline or tetracycline analog.

214. The nucleic acid construct of claim 198, wherein the at least one second promoter is an inducible promoter.

215. The nucleic acid construct of claim 214, wherein the at least one first promoter is a repressible promoter under the control of at least one transcriptional repressor.

216. The nucleic acid construct of claim 215, wherein the inducible promoter is selected from a stress promoter or a heat shock promoter.

217. A method for producing a gene knock down system comprising introducing the nucleic acid construct of claims 198, 199, 200, or 201 into a nonhuman mammal.

218. A method of producing a gene knockdown system comprising:
(a) introducing into a first nonhuman mammal a nucleic acid construct comprising at least one target gene suppressor under the control of at least one promoter under the control of at least one transcription-activating gene;
(b) introducing into a second nonhuman mammal a nucleic acid construct comprising at least one transcription-activating gene under the control of at least one repressible promoter under the control of at least one transcriptional repressor; and
(c) mating the first and second nonhuman mammals to form an offspring mammal having (1) a first nucleic acid construct comprising the at least one target gene suppressor under the control of the at least one promoter under the control of the at least one transcription-activating gene and (2) a second nucleic acid construct comprising the at least one transcription-activating gene under the control of the at least one repressible promoter under the control of the at least one transcriptional repressor.

219. The method of claim 218, wherein at least one promoter in the first nucleic acid construct in the offspring mammal is another repressible promoter also under the control of the at least one transcriptional repressor.

220. The method of claim 219, wherein the at least one transcriptional repressor controlling the at least one repressible promoter and controlling the another repressible promoter are different transcriptional repressors.

221. The method of claim 219, wherein at least one of the repressible promoters is tissue-specific and the at least one target gene suppressor is siRNA.

222. The method of claim 221, wherein the tissue specific repressible promoter is liver-specific.

223. The method of claim 221, wherein the siRNA is ABCA1 siRNA.

224. The method of claim 218, wherein the at least one transcription-activating gene in the first nucleic acid construct of the offspring mammal is a transposon element.

225. The method of claim 218, wherein the at least one transcription-activating gene in the second nucleic acid construct of the offspring mammal is a site-specific DNA recombinase.

226. The method of claim 225, wherein the site-specific DNA recombinase is Cre or Flp.

227. The method of claim 218, wherein the transcriptional repressor in the second nucleic acid construct of the offspring mammal is an induction agent regulated repressor.

228. The method of claim 227, wherein the induction agent regulated repressor is selected from a tetracycline regulated repressor or a tetracycline analog regulated repressor.

229. A method of producing an inducible gene knockdown system comprising:
(a) introducing into a first nonhuman mammal a nucleic acid construct comprising ABCA1 siRNA under the control of a U6 promoter, wherein the ABCA1 siRNA and the U6 promoter are separated by two loxP sites and EGFP coding sequence;
(b) introducing into a second nonhuman mammal nucleic acid construct comprising Cre recombinase gene under the control of a tissue-specific promoter under the control of at least one tetracycline regulated repressor; and
(c) permitting the first and second nonhuman mammals to mate such that the offspring bears a transgene comprising the ABCA1 siRNA under the control of the tissue-specific Cre recombinase under the control of the at least one tetracycline regulated repressor.

230. The method of claim 229, wherein the U6 promoter contains a binding site for the at least one tetracycline regulated repressor.

231. A nonhuman mammal having a transgene made by the method of claim 217.

232. A nonhuman mammal having a transgene made by the method of any of claims 218, 219, or 220.

233. A nonhuman mammal having a transgene of claims 198, 199, 200, 201, 210, or 213.

234. A cell transfected with the nucleic acid construct of claims 198, 199, 200, 201, 210, 213, 214, 215, or 216.
